(19) United States
(12) Patent Application Publication
(10) Pub. No.: US 2024/0260610 A1
(43) Pub. Date: Aug. 8, 2024

(54) ANIMAL FEED ADDITIVE AND METHODS FOR ITS PREPARATION

(71) Applicant: THE CAWTHRON INSTITUTE TRUST BOARD, Nelson (NZ)

(72) Inventors: Michael PACKER, Nelson (NZ); Donato ROMANAZZI, Nelson (NZ); Johan SVENSON, Nelson (NZ)

(21) Appl. No.: 18/562,369

(22) PCT Filed: May 23, 2022
(86) PCT No.: PCT/IB2022/054788

§ 371 Date: Nov. 20, 2023

(30) Foreign Application Priority Data
May 21, 2021 (NZ) 776401
Jun. 18, 2021 (NZ) 777397
Dec. 6, 2021 (NZ) 783108

(51) Int. Cl. A23K 10/30; A01D 44/00; A01G 33/00; A23K 20/105

(52) U.S. Cl. CPC A23K 10/30; A01D 44/00; A01G 33/00; A23K 20/105

(57) ABSTRACT

Animal feed additives comprising the dried biomass of Asparagopsis containing enhanced levels of bromoform are described. Methods of culturing Asparagopsis and processing the harvested biomass that are particularly suited to the preparation of the animal feed additive are also described.

### TECHNICAL FIELD

The invention relates to methods of culturing species of the genus Asparagopsis onshore, methods of establishing offshore farms for the cultivation of the species, and methods of processing the harvested biomass of the species for use as a feed additive for ruminant animals. In particular, the invention relates to methods of culturing Asparagopsis armata, methods of establishing offshore farms for the cultivation of Asparagopsis armata, and methods of processing the harvested biomass of Asparagopsis armata so as to increase its bromoform content.

### BACKGROUND ART

In New Zealand there are two named species of Asparagopsis, a red algae (Rhodophyta) belonging the family Bonnemaisoniaceae. Asparagopsis armata is commonly found around New Zealand, whereas Asparagopsis taxiformis has a more limited distribution in New Zealand waters (Kermadec Islands). Asparagopsis armata is also found as an introduced species in Europe where it has spread widely.

Asparagopsis armata grows epilithicly or epiphytically in low intertidal or subtidal pools. Varying from pale pink to red in colour, Asparagopsis armata has two morphologically different stages of development—the gametophyte stage and the tetrasporophyte stage. The gametophyte stage is the more conspicuous stage growing up to 25 cm long with a cylindrical main axis and many radially arranged feathery branchlets.

In the wild the tetrasporophyte stage may be free-floating or grow as a soft, low turf on rock, structures or as an epiphyte on seaweeds (including the gametophyte). Looking like pink to red cotton wool or “pom poms”, the filaments of the tetrasporophyte stage comprise three cells around each axial cell, a domed apical cell and gland cells. The filamentous tetrasporophyte stage was initially described as a different species (Falkenbergia rufolanosa) but the life history connection between the morphologically distinct stages of development is now understood.

Interest in the culture and cultivation of species of Asparagopsis has arisen from its potential for use as an inhibitor of methanogenesis when included in the feed of ruminant animals. This use is attributed to the presence of halogenated compounds such as bromoform in the biomass of the species.

The publication of Lanigan (1972) discloses the in vivo inhibition of methane formation in the sheep's rumen by the compound bromoform and the publication of Paul et al (2006) discloses that bromoform is a major (0.58 to 4.3% of dry weight) natural product in both life-history stages of Asparagopsis armata.

The publication of Lognone et al (2005) discloses methods for the cultivation of red algae of the Bonnemaisoniaceae family. The methods use the tetrasporophytic form of the algae. The methods are presented as an improvement over existing methods of cultivation, e.g., on lines at sea. The publication asserts that the methods disclosed allow for the production of algal biomass on land, under intensive conditions, while controlling its content of halogenated compounds and, consequently, to use this biomass for the production of an antibiotic extract whose activity is standardized. Exposure of the cultures to light is controlled by various means such as the depth of the ponds, the density of the culture and the agitation.

The publication of Tomkins et al (2009) discloses the use of a formulation of bromochloromethane (BCM) to reduce enteric methanogenesis in cattle. The manufacture and use of BCM was banned in Australia after completion of the reported studies and the publication concludes that it is unlikely that the BCM formulation will be available for commercial use for methane mitigation in Australia's agricultural sector. The publication suggests that the future use of alternative antimethanogens, with a similar mechanism of action, may have practical commercial relevance given that cobamide-dependent methane production had been demonstrated to be inhibited for up to 90 days, the period typically used for feedlot finishing of cattle.

The publication of Dubois et al (2013) discloses the inclusion in ruminant diets of marine and freshwater algae (green and red) for the management of greenhouse gas emissions. Referencing the disclosures of the publication of Paul et al (2006), the publication states that the effects induced by each algal species will depend on their composition and the nature, activity and concentration of its bioactive components.

The publication of Machado et al (2014) discloses that of twenty species of macroalgae, species of Dictyota and Asparagopsis have the strongest effect on methane production in in vitro studies, stating that the study provides the first evidence that macroalgae can effectively reduce in vitro methane production as all species had similar or lower TGP and CH₄ production to a positive control of decorticated cottonseed (DCS). The publication further states that given the significant effects of Asparagopsis in reducing total gas production and CH₄ output, it is likely that lower doses of this alga can now be targeted to reduce CH₄ output without affecting the nutritionally important fermentation parameters.

The publication of Machado et al (2015) provides additional data from in vivo studies (Example 4 and Example 5). The method for reducing total gas production and/or methane production in ruminant animals comprises the step of administering to the animals an effective amount of a red marine macroalgae. A species of Asparagopsis, e.g., Asparagopsis taxiformis, is identified as a preferred red marine macroalgae. The inhibition is reported to occur at concentrations below that at which the degradation of dry matter is inhibited.

The publication of Tomkins et al (2018) discloses a method for improving growth performance of a livestock animal in pasture and feedlot farming systems. The method includes feeding the livestock animal a red marine macroalgae such as wild harvested Asparagopsis taxiformis. The harvested biomass is frozen immediately to prevent loss of bromoform. Improvements in average growth rates of 0.1 and 0.3 Kg per day are suggested.

The publication of Sawant (2019) discloses algal composition in the form of an aqueous suspension. The algae may be a species of Asparagopsis such as Asparagopsis armata or Asparagopsis taxiformis. The compositions are intended for use in improving plant health. The results of field studies on cucumber, green gram, maize and tomato are presented.

The publication of De Nys et al (2020) discloses a method of extracting bromoform from the biomass of Asparagopsis to provide a composition suitable for reducing methanogenesis in a ruminant animal.

The publication of Goldman and Mata (2021) discloses a bioreactor for use in culturing seaweed, in particular Asparagopsis armata or Asparagopsis taxiformis. The bioreactor is characterised by a porous barrier separating a first compartment where sporophytes are retained and a second compartment into which spores may pass. As noted above, the publication acknowledges that there is a need for a system and method for culturing seaweed species to be used in cattle feeds, serving as a means for reducing greenhouse gas emissions from farm-raised cattle. The method disclosed in the publication comprises reducing the hours of daily light to which the sporophytes are exposed, thereby inducing sporogenesis, and in turn, inducing the spores resulting from this sporogenesis to attach to one or more settlement surfaces. However, the publication does not disclose how an axenic culture of the desired species of Asparagopsis is first established.

It is an object of the present invention to provide a method of producing biomass of a species of Asparagopsis. It is an object of the present invention to provide a method of increasing the bromoform content of the harvested biomass of a species of Asparagopsis. It is an object of the present invention to provide a method of establishing a farm for the cultivation of a species of Asparagopsis. It is an object of the present invention to provide an animal feed additive comprising biomass of a species of Asparagopsis. The foregoing objects are to be read in the alternative with the object at least to provide a useful choice.

### SUMMARY OF INVENTION

In a first aspect an animal feed additive is provided, the feed additive comprising biomass of a species of Asparagopsis where the bromoform content of the biomass is greater than 5 mg/g. The feed additive is therefore distinguished from an unrefined extract. The bromoform content of the biomass is determined according to the method of determining bromoform content described herein and expressed as mg of bromoform per g of wet weight of harvested biomass. Preferably, the bromoform content of the biomass is greater than 7 mg/g. More preferably, the bromoform content of the biomass is greater than 14 mg/g. Most preferably, the bromoform content of the biomass is greater than 21 mg/g. Preferably the biomass is in the form of a powder, such as may be produced by freeze-drying. Preferably the biomass is substantially purple in colour. Preferably the biomass consists essentially of tetrasporophytes. Preferably the species of Asparagopsis is Asparagopsis armata.

In a second aspect a method of culturing a species of Asparagopsis to produce biomass for use in the preparation of an animal feed additive is provided, the method comprising the incubation of a volume of seawater containing propagules of the species of Asparagopsis under green light. In a first alternative the green light is filtered daylight. In a second alternative the green light is provided by an artificial light source, e.g., one or more light emitting diodes. The propagules of the species of Asparagopsis are maintained in suspension. Preferably, the propagules of the species of Asparagopsis are maintained in suspension by circulating the seawater. Preferably, the propagules are tetrasporophytes. Preferably, the volume of seawater is amended with nutrient F-medium containing 0.5 mg/L germanium dioxide. Preferably, the green light has a median wavelength in the range 475 to 580 nm. More preferably, the green light has a median wavelength of around 525 nm.

In a third aspect a method of preparing an animal feed additive is provided, the method comprising harvesting biomass from a culture of a species of Asparagopsis and subjecting the biomass to a physical stress that is sufficient to produce at least a two-fold increase in the bromoform content of the biomass. Preferably, the subjecting the biomass to a physical stress, sufficient to produce a change in colour of the biomass from red to purple. Preferably, the physical stress comprises compression, exposure to sunlight, or partial desiccation of the biomass. More preferably, the physical stress comprises both compression and partial desiccation of the biomass. Preferably, the ratio of total bromine to iodine content of the biomass is increased.

In a preferred embodiment a method of preparing an animal feed additive is provided, the method comprising the incubation of a volume of seawater containing tetraporophytes of the species of Asparagopsis under green light to provide a culture, harvesting biomass from the culture, subjecting the biomass to at least partial desiccation so as to cause the biomass to develop a purple colour, and then freezing the biomass.

In a fourth aspect a method of inducing tetrasporophytes of Asparagopsis armata to produce tetraspores is provided. The method comprises reducing the temperature and modifying the alternating light-dark schedule of an established culture of the tetrasporophytes. The temperature of the established culture is reduced to 12 to 14° C. and the alternating light-dark schedule is modified to 6 hours green light and 18 hours dark. Preferably, the established culture has been maintained at a temperature of 21 to 25° C., more preferably 22 to 24° C., with an alternating light-dark schedule of 12 hours green light and 12 hours dark. More preferably, the circulating of the established culture is maintained during the reducing the temperature and modifying the alternating light-dark schedule.

In a fifth aspect a method of producing a substrate seeded with inchoate gametophyes of a species of Asparagopsis is provided. The substrate is for use in establishing offshore farms for the cultivation of the species of Asparagopsis. Preferably, the species of Asparagopsis is Asparagopsis armata. The method comprises contacting a culture of tetrasporophytes induced to produce tetraspores according to the fourth aspect with a substrate to be seeded and incubating at a temperature and for a time sufficient to provide the seeded substrate.

Preferably the substrate is selected from those disclosed in the publication of Goldman and Matta (2021).

In a sixth aspect a nutritional preparation for an animal comprising an amount of the feed additive of the first aspect, an amount of biomass prepared according to the second aspect, or a feed additive prepared according to the method of the third aspect is provided. The amount of the biomass or feed additive is an amount effective to reduce the total methane production in the population of ruminants to which the nutritional preparation is to be fed. The reduction in the total methane production is relative to an equivalent population of ruminants to which the nutritional preparation is not fed. Methods for determining the total methane production in a ruminant are disclosed in the publication of Machado et al (2015).

In a seventh aspect a method of reducing the total methane production in a population of ruminants is provided. The method comprises feeding to the population a nutritional preparation of the sixth aspect.

In all the aspects and embodiments provided the animal is a ruminant, i.e., a mammal of the order Artiodactyla. Preferably, the mammal of the order Artiodactyla is of the family Bovidae. More preferably, the mammal of the order Artiodactyla of the family Bovidae is of the subfamily Bovinae. Yet more preferably, the mammal of the order Artiodactyla of the family Bovidae of the subfamily Bovinae is of tribe Bovini. Yet even more preferably, the mammal of the order Artiodactyla of the family Bovidae of the subfamily Bovinae of tribe Bovini is of the genus Bos. Most preferably, the mammal of the order Artiodactyla of the family Bovidae of the subfamily Bovinae of tribe Bovini of the genus Bos is the species Bos taurus.

In the description and claims of this specification the following abbreviations, acronyms, phrases and terms have the meaning provided: “amend” means to change or improve, e.g. by the addition of nutrients; “axenic” means a culture that is substantially free from living organisms other than the species required; “bromoform” means tribromomethane (CHBr₃) (CAS RN 75-25-2); “CAS RN” means Chemical Abstracts Service (CAS, Columbus, Ohio) Registry Number; “comprising” means “including”, “containing” or “characterized by” and does not exclude any additional element, ingredient or step; “consisting essentially of” means excluding any element, ingredient or step that is a material limitation; “consisting of” means excluding any element, ingredient or step not specified except for impurities and other incidentals; “cultivate” means grow on a large scale; “culture” means grow in a medium containing nutrients; “epifauna” means animals living on the surface of the seabed or a riverbed, or attached to submerged objects or aquatic animals or plants; “farm” means an area for the cultivation of plants or rearing of animals; “feed additive” means a non-nutrient substance added to the feed of animals to improve the preservation, digestion, colour, palatability, texture, or nutritive value of the feed; “feed” means edible material that both provides nourishment in the form of energy and for building tissues and contributes to the normal physiological function and metabolic homeostasis of an animal; “F-medium” means medium f as defined in Table II of the publication of Guillard and Ryther (1962); “green light” means light having a median wavelength in the range 475 to 580 nm; “inchoate” means not fully formed or developed; “infauna” means the animals living in the sediments of the ocean floor or river or lake beds; “LOD” means limit of detection, i.e., the lowest concentration of analyte in a sample that can be detected; “LOQ” means limit of quantitation, i.e., the lowest concentration of analyte in a sample that can be accurately quantified; “LOR” means limit of repeatability/reproducibility, i.e., the lowest concentration of analyte in a sample that can be reliably determined both within and between laboratories; “offshore” means situated at sea some distance from the shore; “onshore” means situated or occurring on land; “nutritional preparation” means a compounded mix of animal nutrients or animal nutrients and feed additives; “population” means a group of individuals of the same species; “propagules” means a vegetative structure that can become detached from a plant and give rise to a new plant; “RSD,” is a measure of the repeatability of a method assessed according to ISO 5725-2 (1994); “RSDR” is a measure of the reproducibility of a method assessed according to ISO 5725-2 (1994); and “unrefined extract” means plant material that has not been subjected to purification processes that result in the isolation of, or alteration of the proportions of, specific chemical constituents of the plant. A paronym of any of these defined terms has a corresponding meaning.

The terms “first”, “second”, “third”, etc. used with reference to elements, features, integers or other limitations of the matter described in the Summary of Invention, or when used with reference to alternative aspects or embodiments are not intended to imply an order of preference. Preferments for elements, features, integers or other limitations described in the Summary of Invention are identified in order of preference by the introductory “preferably . . . ”, “more preferably . . . ”, “most preferably . . . ”, and so on. Preferred combinations of elements, features, integers or other limitations described in the Summary of Invention are similarly identified.

Where concentrations or ratios of reagents are specified the concentration or ratio specified is the initial concentration or ratio of the reagents. Similarly, where a pH or range of pH is specified, the pH or range of pH specified is the initial pH or range of pH. Where values are expressed to one or more decimal places standard rounding applies. For example, 1.7 encompasses the range 1.650 recurring to 1.749 recurring. Unless otherwise stated, concentrations of bromoform are expressed as mg per g of wet weight of biomass.

The invention will now be described with reference to embodiments or examples and the figures of the accompanying drawings pages.

### BRIEF DESCRIPTION OF DRAWINGS

FIG. 1A. Analysis by GC-MS of bromoform analytical standard (>988) at a concentration of 2.5 μg/mL.

FIG. 1B. Analysis by GC-MS of a freeze-dried sample of biomass (Asparagopsis armata).

FIG. 1C. Analysis by GC-MS of a fresh sample of biomass (Asparagopsis armata).

FIG. 2. Initial scheme for the culturing, harvesting and processing of biomass of Asparagopsis armata to provide an animal feed additive.

FIG. 3. Photograph of an isolated tetrasporophyte of Asparagopsis armata (“pom pom”).

FIG. 4. Schematic top plan (A) and side perspective (B) views of green plastic buckets used in preparation of inoculum.

FIG. 5. Schematic top plan view of a green plastic bucket held partially submerged in a parabolic tank by means of a rigid holding frame placed over the tank.

FIG. 6. Side (A and B) and end (C and D) views of parabolic tanks covered with lid.

### DESCRIPTION

Bromoform and chemically related compounds are naturally produced in green, brown, and red marine macroalgae. However, some of the highest concentrations of bromoform are produced in the red macroalgal genus Asparagopsis. Recently, Asparagopsis taxiformis has been successfully used to inhibit the production of enteric methane in ruminants in in vivo studies and methane production from rumen microbe assemblages in vitro. However, there are several obstacles to the use of the biomass of a species of Asparagopsis as a feed additive to reduce methanogenesis in ruminant animals.

Firstly, the biomass must be available in sufficient quantity to meet the demand and desirably be of consistent quality. Wild harvesting of the biomass may not be sustainable and introduces uncertainty as regards the quantity of biomass available throughout the year. Secondly, the biomass must be free of phycotoxins that would preclude the use of the biomass for the intended purpose. For example, contamination with neurotoxins produced by certain species of microalgae or cyanobacteria might result in harm to the health of the ruminant animal to which the biomass was fed. Thirdly, the biomass used in the preparation must desirably be of consistent quality to minimise batch to batch variation and the amount of feed additive to be used to reduce methanogenesis consistently. Ideally, the regular feed of the ruminant animal needs to be amended with a minimal amount of the feed additive.

These obstacles may be overcome by culturing a species of Asparagopsis under conditions that mitigate the risk of contamination with neurotoxins and processing the harvested biomass so as to enhance the bromoform content, the constituent to which the reduction in methanogenesis is attributed. It has been found that when culturing Asparagopsis armata in parabolic tanks located outdoors the production of biomass is favoured, and the growth of cyanobacteria discouraged, by modifying the incident daylight. This modification may be achieved by interposing a green filter such as a translucent sheet of green plastic. It is recognised that the required modification of the incident light may also be achieved by selecting an appropriate light source with an emission spectrum centred at the desired wavelength. Other alternatives include the use of coloured meshes or screens that serve to both scatter and/or filter the incident light. According to the methods described here, free-floating tetrasporophytes of Asparagopsis are maintained in a continuous culture with periodic harvesting of the biomass and replenishment of the seawater. It has been discovered that by submitting the harvested biomass to a physical and/or physiological stress such as partial desiccation or compaction, a substantial (greater than two-fold) increase in the bromoform content of the biomass can be obtained. This increase correlates with a change in colour of the harvested biomass from red to purple and is a useful indicator of the quality of the biomass when preparing the feed additive. The colour change approximates to a change from PANTONE™ 1815C to PANTONE™ 235C when viewed under natural light.

### Preparation of Amended Seawater

UV sterilized, filtered seawater is collected in 1 L bottles (Schott) and autoclaved. F/2 (1, 000× concentrate) (ALGABOOST™) is filtered (0.22 μm, 47 mm (Millipore)) and stored frozen at a temperature of −20° C. without exposure to light. A solution of germanium dioxide (GeO₂) at a concentration of 1 g/L in deionized water (MilliQ) is filtered (0.22 μm, 47 mm (Millipore)) prior to use. In a laminar flow hood the required volumes of the F/2 (1,000× concentrate) (ALGABOOST™) and solution of germanium dioxide (GeO₂) are added to a volume of the autoclaved seawater to provide F/8 media containing a final concentration of 50 mg/L of GeO₂. Sealed bottles of this amended seawater are stored at room temperature prior to use in the culturing of tetrasporophytes of Asparagopsis armata.

### Culture of Tetrasporophytes—Initial Studies

Initially, a few grams of wild harvested free floating tetrasporophytes of Asparagopsis armata (North Auckland or Tory Channel separately, New Zealand) were used to inoculate a volume of 5 L of seawater amended with a 2 to 8-fold dilution of F-medium. The inoculated volume was incubated at ambient temperatures and daylength until multiplication was observed. A quantity of 3 to 128 g (wet weight) of the growing inoculum was then used to inoculate a volume of 3,000 L of the seawater. The inoculated volume was held in an opaque walled parabolic bath located outside and fitted with a circulating water pump to maintain the tetrasporophytes in suspension. The bath was covered with a translucent green filter to modify the quality of the incident daylight. The culture was maintained from late December to April during which time the ambient temperature varied between 6 and 23° C. Quantities of the multiplying tetrasporophytes were periodically harvested by removing a portion of the volume and sieving to collect the biomass. The volume in the bath was periodically replenished with the amended seawater to maintain the culture.

Indicative yields and productivities based on these initial studies are presented in Tables 1 and 2.

TABLE 1
Indicative yields of biomass based on initial studies.

| | | | Initial time | | Final time | Initial wet | | Final wet | |
|---|---|---|---|---|---|---|---|---|---|
| | | | point | | point | weight | | weight | |
| Vessel | Volume | | (t₁) | | (t₂) | (N₁) | | (N₂) | |
| Bucket | 16 | L | 7 | day | 13 day | 6.47 | g | 12.79 | g |
| Parabolic | 3,000 | L | 0 | day | 12 day | 12.0 | g | 185.6 | g |
| tank | | | | | | | | | |
| Parabolic | 3,000 | L | 12 | day | 22 day | 185.6 | g | 719.6 | g |

tank

TABLE 2
Productivity based on initial studies.

| | | | | Wet | Wet weight |
|---|---|---|---|---|---|
| | | | Time | weight | per litre |
| Vessel | Volume | | period | per day | per day |
| Bucket | 16 | L | 7 to 13 days | 1.053 | 0.07 |
| Parabolic tank | 3,000 | L | 0 to 12 days | 14.467 | 0.005 |
| Parabolic tank | 3,000 | L | 12 to 22 days | 53.400 | 0.02 |

### Determination of Bromoform Content

### Materials

Methanol (analytical grade); dry ice (CO₂); bromoform (analytical standard, >98%) (Sigma-Aldrich).

### Standards

A stock solution of bromoform (analytical standard) was prepared at a concentration of 5,000 μg/mL by accurately weighing a quantity of 50 mg (+/−2.0 mg) into a volumetric flask and making up to a total volume of 10 mL with methanol. The stock solution was stored at a temperature of minus 18° C. for a period of time no greater than 3 months. Calibration standards (0.025, 0.25, 0.5, 1 and 2.5 μg/mL) were prepared as required by serial dilutions of the stock solution using methanol as diluent and used within one month.

### Method

Frozen samples of a quantity of about 1 g were allowed to thaw slightly and homogenised with dry ice in a blender to provide a free-flowing powder. The homogenised sample was then stored in a freezer to allow evaporation of any residual carbon dioxide (CO₂). An amount of about 1 g (±0.1 g) of a homogenized sample was weighed into a 15 mL polyethylene (PE) tube and the precise weight measured to within two decimal places. A volume of 10 mL of methanol (MeOH) was added to the tube and the contents sonicated in an ice water bath for a period of time of 30 minutes before centrifugation (3,000×g) for a period of time of 10 minutes. The methanolic supernatant was transferred to a 50 mL PE tube and the extraction repeated. The volumes of methanolic supernatant were combined, mixed and an aliquot having a volume of 100 μL diluted to a final volume of 10 mL with methanol for analysis by gas chromatography-mass spectrometry (GC-MS).

Amounts of about 100 mg (±10 mg) of freeze-dried samples of biomass were weighed into a 15 mL PE tube and the precise weight measured to within one decimal place. A volume of 10 mL of methanol (MeOH) was added to the tube and the contents sonicated in an ice water bath for a period of time of 30 minutes before centrifugation (3,000×g) for a period of time of 10 minutes. The methanolic supernatant is transferred to a 50 mL PE tube and the extraction repeated. The volumes of methanolic supernatant are combined, mixed and an aliquot having a volume of 100 μL diluted to a final volume of 10 mL with methanol for analysis by gas chromatography-mass spectrometry (GC-MS).

Diluted samples were analysed by GC-MS using an Agilent 7890B/5977A Series Gas Chromatograph (GC)/Mass Selective Detector (MSD) fitted with microsplitter and an Agilent 19091N-133 HP-INNOWAX column (30 m, 0.25 mm, 0.25 μM). A volume of 1 μL of the diluted sample was injected at an inlet temperature of 180° C. and pressure of 9.8 psi. Column gas flow was 1.5 mL/min

TABLE 3
Operating parameters for MSD.

| MS Source temperature | 230° | C. |
|---|---|---|
| MS Quad temperature | 150° | C. |
| MSD mode | SIM | |
| Quantifier m/z | 172.8 | |
| Qualifiers m/z | 170.8, 174.8, 251.8, 253.8 | |
| Dwell time | 50 | ms |
| Solvent delay (MSD on) | 4 | min |
| Timed events (MSD off) | 13 | min |

with an oven temperature commencing at a temperature of 40° C. for one minute before increasing to a temperature of 250° C. at a rate of 16° C./min and held at 250° C. for a period of time of two minutes for a total run time of 16.25 minutes. With a transfer line temperature of 280° C. the MSD was operated according to the parameters provided in Table 3.

A series of samples was analysed with intermittent inclusion of a calibration standard no less frequently than once in every six analyses. Data acquisition and quantitative analysis were performed using Agilent MassHunter™ software according to the following equation:

$$Bromoform Content ( mg / g ) = \frac{Bromoform result \times Sample Vol \times Dilution}{Sample Wgt ( g ) \times 1000}$$

where ‘Bromoform result’ is the result (μg/mL) calculated by the Agilent MassHunter™ software; ‘Sample Vol’ is the sample volume (mL), i.e., 20 mL; ‘Dilution’ is the final dilution of the sample (typically 100); and ‘Sample Wgt’ is the weight (g) of sample used.

Assay performance characteristics based on the single laboratory validation results are summarised in Table 4.

TABLE 4
Assay performance.

| | Detection Limit | | | Precision | | Accuracy |
|---|---|---|---|---|---|---|
| Assay | LOD | LOQ | LOR | Repeatability | Reproducibility | Recovery |
| Bromoform | (mg/g) | (mg/g) | (mg/g) | RSDᵣ | RSD_{R} | from matrix |
| Fresh | 0.04 | 0.14 | 0.14 | 5.0% | 8.5% | 98.8% |
| biomass | | | | | | |
| Freeze- | 0.40 | 1.40 | 1.40 | 5.8% | 9.0% | |
| dried | | | | | | |
| biomass | | | | | | |

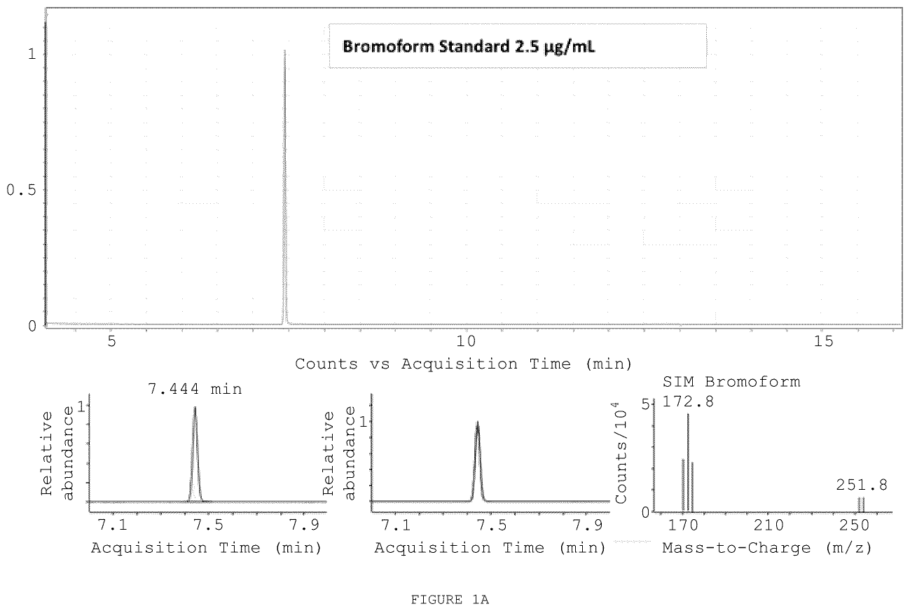

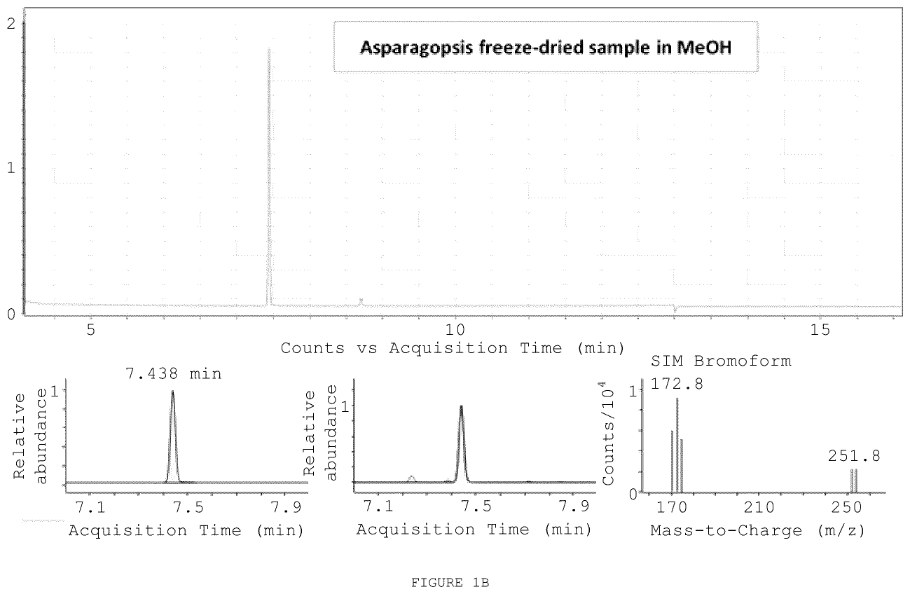

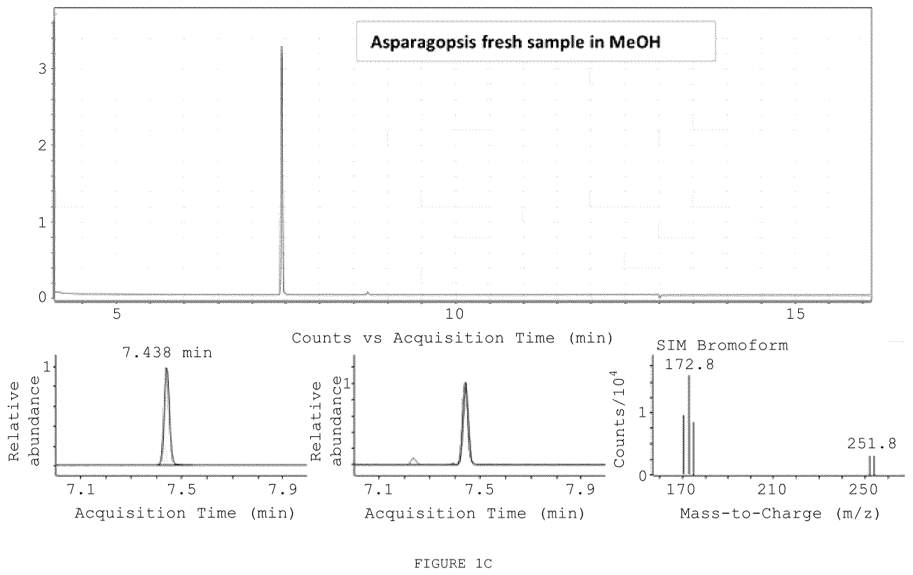

Selectivity of the analytical method was confirmed by a comparison of the retention times, MS data and Q/q confirmation ratios acquired for bromoform (analytical standard, >98%) (Sigma-Aldrich) and freeze-dried or frozen samples of harvested biomass (FIGS. 1A-C). Confirmation ratios of the bromoform signal in the analytical standard and samples were in good agreement. The MS spectra and MS data for the bromoform peak in the samples matched those from the analytical standard. The retention time for bromoform in the analytical standard was consistent with the retention time in the samples (±2%). No peaks were seen to co-elute with bromoform so as to interfere with repeatable quantitation.

### Harvesting and Processing Biomass

A volume of an established (over two weeks old) culture of Asparagopsis armata was collected and the bromoform content of the collected biomass periodically determined while the biomass remained in seawater for a period of time of 90 minutes. The biomass was then separated from the seawater by transferring to a sieve and the bulk of the retained seawater expelled by compressing the biomass. This harvested biomass was then allowed to dry so as to produce a change in colour of the biomass from red to purple. The bromoform content of the harvested biomass was periodically determined throughout. The results of the determinations are summarised in Table 5.

TABLE 5
Bromoform content and colour of biomass
during different stages of harvesting.

| | Time | Bromoform content | |
|---|---|---|---|
| Stage | (minutes) | (mg/g) | Colour |
| In seawater | 0 | 3.41 | Red |
| | 30 | 3.14 | |
| | 60 | 2.10 | |
| | 90 | 2.16 | |
| Compressed on | 100 | 7.12 | Red-purple |
| sieve | 120 | 6.03 | |
| | 130 | 6.55 | Purple |
| | 160 | 4.99 | |
| | 190 | 7.17 | |

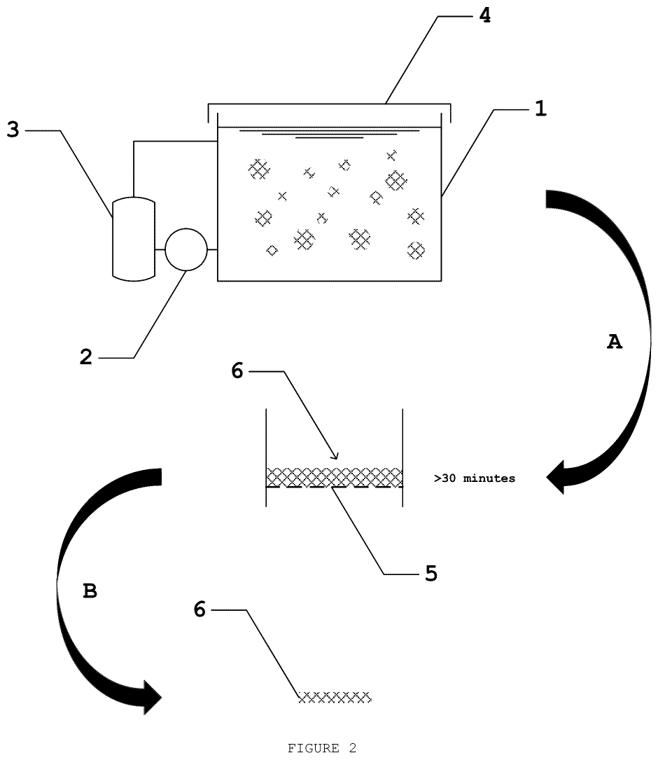

Referring to FIG. 2 a proposed scheme for the manufacture of a feed additive is presented. Tetrasporophytes of Asparagopsis armata are cultured in natural seawater amended with F-medium and germanium dioxide in a covered tank (1). The tetrasporophytes are maintained in suspension by means of a circulating water pump (2) and secondary tank (3) via which seawater removed from the covered tank (1) may be replenished. The tank (1) is covered with a green translucent filter (4) to modify the incident light, removing red light and decreasing the total photon flux. According to Step A volumes of the culture are periodically removed from the covered tank (1) and transferred to a sieve (5) to separate the biomass (6) from the seawater. The separated biomass (6) is then compressed to expel retained seawater and allowed to air dry for a minimum period of 30 minutes until the biomass (6) develops a purple colour. According to Step B the bromoform content of the partially desiccated biomass (6) is determined and the biomass (6) then frozen. The frozen biomass (6) is then freeze-dried directly or allowed to partially thaw before mixing with dry ice. In either case the dried biomass is ground to a powder and the bromoform content determined. Where necessary, the bromoform content may be adjusted by blending with similarly prepared biomass of Asparagopsis armata, but not subjected to partial desiccation. Accordingly, a powdered feed additive of consistent bromoform content, e.g., 6 to 8 mg/g, can be supplied. The bromoform content of the feed additive will typically be 80 to 90% greater than that of freshly harvested biomass thereby reducing packaging, storage and transport costs.

### Culture of Tetrasporophytes—Scale-Up

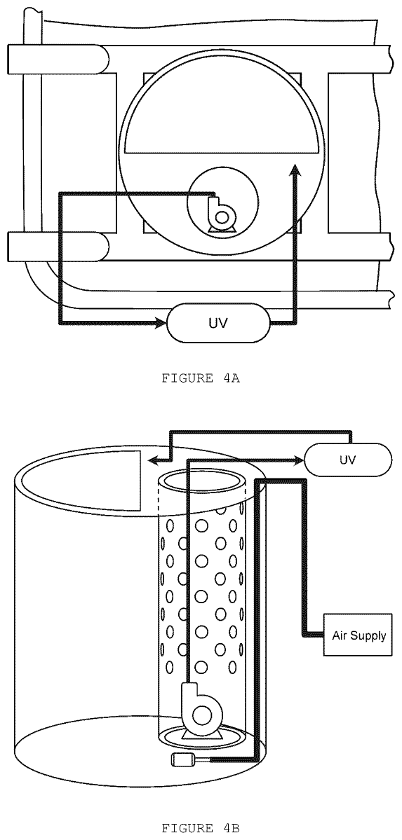

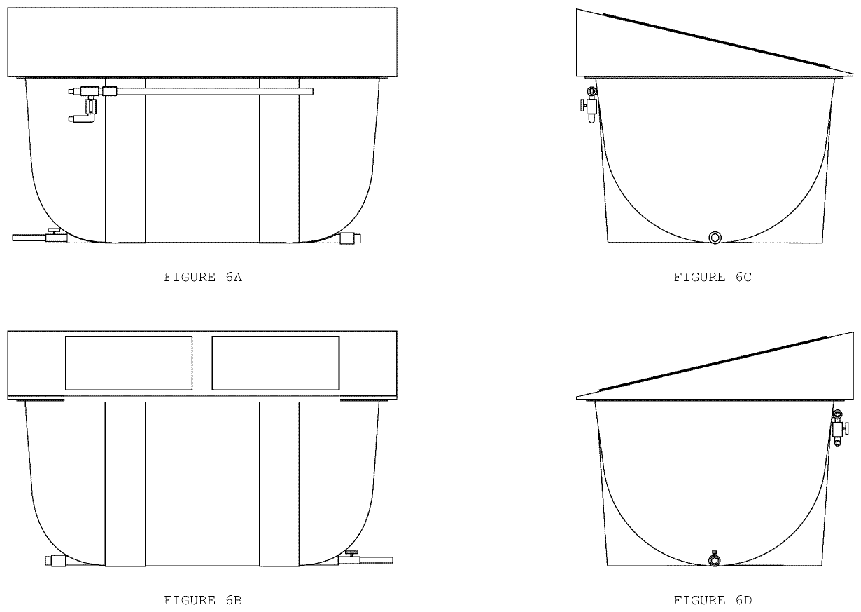

Following these initial studies procedures for the culture of larger volumes of tetrasporophytes were developed and are described in more detail with reference to FIGS. 4 to 6 of the accompanying drawings pages.

Isolation of Asparagopsis armata

### From Carposporophytes

Gametophytes of Asparagopsis armata that contained ripe carposporophytes were collected from the wild in November (Summer Hemisphere early summer).

Cuttings of 2 to 3 cm length were trimmed from the fertile cystocarp-bearing fronds. Each of the cuttings was then transferred to a well of a 6-well plate containing the amended seawater (F/8 containing 0.5 mg/L GeO₂) and incubated at a temperature of 21° C. The plates were illuminated with green light (525 nm±10 nm) from light emitting diodes (LEDs) at an irradiance of 30 μmol m⁻² s⁻¹ with 12 hours light; 12 hours night period. The wells of the 6-well plates were periodically inspected under a microscope for the release of spores. Once the release of spores had occurred the plates were incubated at both temperatures of 21° C. and 13° C. at the same 30 μmol m⁻² s⁻¹ with 12 hours light; 12 hours night period. The released spores were observed to germinate after about a day and adhere firmly to the bottom of the well. The lower temperature assisted with establishment of new tetrasporophyte tissue as contaminating organisms grew slower. Once tetrasporophyte filaments were present, vegetative growing tips were carefully excised and placed in new plastic pottles with the same growth medium. Once clean tetrasporophyte cultures had been established the temperature was gradually increased to 21° c.

### Tetrasporophytes

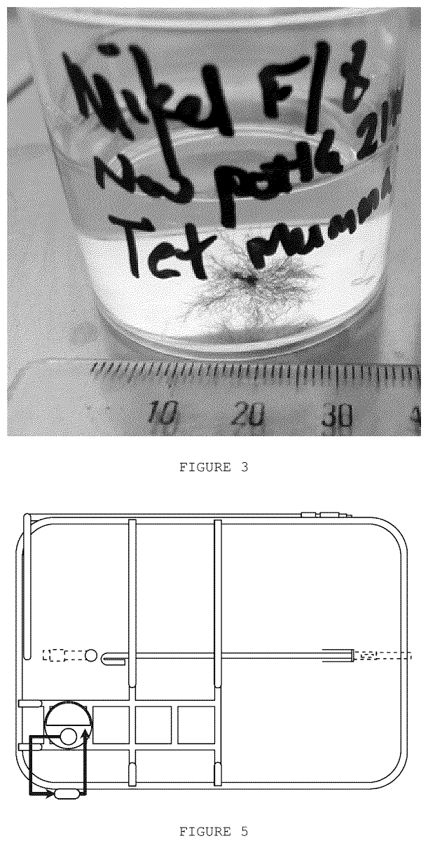

Cultures of tetrasporophytes were maintained in the amended seawater as individual small fragments or larger “pom poms” (FIG. 3) in 70 ml polystyrene pottles. The cultures were maintained in a cabinet at a temperature of 21° C. and illuminated according to a 12-hour alternating light-dark schedule with green light (525 nm±10 nm) from LEDs with an irradiance of 30 μmol m⁻² s⁻¹. The tetrasporophytes were periodically (monthly) transferred to a new pottle half filled with fresh amended seawater. Larger tetrasporophytes (“pom poms”) were divided between new pottles using tweezers. All transfers were performed in a laminar flow hood to minimise the risk of contamination.

### Preparation of Inoculum

Fragments of tetrasporophytes from multiple pottles were transferred to a 3 L Erlenmeyer flask and the resulting combined volume aerated while being maintained in a cabinet at a temperature of 21° C. and illuminated according to a 12-hour alternating light-dark schedule with green light (525 nm±10 nm) from LEDs with an irradiance of 30 μmol m⁻² s⁻¹.

After two weeks the contents of the flask were transferred to a green plastic bucket (7) containing 16 litres of amended seawater. The contents of the bucket were aerated using an air stone (8) and the seawater recirculated via a pump (9) through a UV sterilizer (10). The inlet hose of the UV sterilizer was fitted with an 80 mm diameter rigid pipe (11) having holes in its wall and wrapped with a 112 micron mesh. The pipe was placed vertically in the contents of the bucket (7) thereby allowing for the retention of the tetrasporophytes in the bucket while the seawater was recirculated via the pump. The recirculation of the UV sterilised seawater serves both to separate potentially contaminating diatoms from the tetrasporophytes and fragment the tetrasporophytes thereby promoting the propagation of an axenic culture.

Multiple buckets (6) were held partially submerged in a 3,000 L parabolic tank by means of a rigid holding frame (12) placed over the tank (1). The tank (1) was filled with seawater to moderate fluctuations in the ambient temperature. The seawater in each of the buckets (7) was periodically (typically every two weeks) replaced with fresh amended seawater and the wet weight of the biomass determined at this time. The buckets (7) and associated tubing were thoroughly cleaned with ethanol and rinsed with freshwater before replacing the seawater. The wet weight was determined by draining the contents of each bucket (7) through a pre-weighed 43 μm sieve (5) and removing excess water by very gentle compression of the retained biomass (6). Once weighed the biomass was immediately transferred to the fresh amended seawater to avoid stressing the tetrasporophytes.

### Culture of Tetrasporophytes

From 13 to 30 g wet weight of the biomass was used to inoculate amended seawater held in a specially modified 3,000 L parabolic tank (1). The seawater was filtered to 1 μm via a series of bag and cartridge filters. The tank was modified by incorporation of a line of aero hose (a.k.a. “soaker hose”) (13) running three quarters of the length of the bottom of the tank (1) and attached to the inside wall. The open end of the hose was connected to a polyethylene pipe (14) connected to an air supply inlet (15) external to the tank. Pressurised air was supplied to the hose (13) to aerate the contents of the tank and ensure continuous circulation of the tetrasporophytes within the volume of seawater held in the tank. The circulation prevented settlement on the internal walls of the tank and promoted propagation of the tetrasporophytes. The tank was additionally provided with an internal standpipe (16) stood vertically in the volume of seawater and having an open mouth at its upper end covered with a 112 micron mesh filter assembly. Biomass was retained while seawater could pass through the assembly and down into a sump from where it was then pumped to a UVC sterilizer and filter (HAILEA™ G8000) before being returned to the tank. The return flows were regulated by 2 ball valves controlling the flow to the tank or back to the sump. During operation the pH of the circulating seawater averaged 8.2 and the dissolved oxygen levels were 1028. Representative samples for determining biomass density were obtained by siphoning or use of a modified 20 L screw lid bucket. The bottom of the bucket was removed, and holes made in the side walls and screw lid. The holes were covered with a 200 μm mesh and handles affixed to the outer walls of the bucket. The lidless bucket was immersed in the contents of the tank and rotated through 90 degrees. The lid was screwed on and the bucket rotated through a further 90 degrees and withdrawn from the tank. Periodically (typically every month) the biomass from a tank was collected using a 200 μm net and transferred to a new tank containing fresh filtered amended seawater. The seawater was then treated with bleach (7 L) for two hours, drained and thoroughly rinsed with freshwater before reuse. During cultivation of the tetrasporophytes the 3,000 L parabolic tanks was either covered with a lid or uncovered. When covered the lid was provided with green plastic windows (4) that served to modify the quality of the incident daylight in terms of both intensity and wavelength.

### Harvesting of Biomass

### Trial 1

The entire biomass contained in an uncovered tank was harvested by a combination of draining through a mesh and collecting with a net. The harvested biomass was held in a bucket in the shade for 90 minutes before being compressed to remove a substantial portion of the entrained water and placed in deep trays in the shade for 60 minutes to air dry. A subsample of the originally harvested biomass was also transferred to a tube and warmed by immersing in boiled water. A total yield of 13.6 Kg (wet weight) of biomass was harvested from the tank.

### Trial 2

Prior to harvest 819.8 g fresh weight of the biomass contained in a covered tank was used to inoculate a fresh 3,000 L parabolic tank containing amended seawater. The remaining biomass was harvested as for Trial 1. The harvested biomass was observed to be darker in colour than that harvested in Trial 1.

The intensity of this colour (dark purple) was observed to be increased by air drying of the biomass and exposure to daylight, in particular direct sunlight. Samples for determination of bromoform content were frozen at −20° C. A total yield of 2.135 Kg fresh weight (following removal of entrained water) was harvested from the tank.

### Trial 3

The entire biomass contained in a covered tank was harvested as for Trial 1. A total yield of 3 Kg fresh weight (following removal of entrained water) was harvested from the tank.

### Preparation of Seeded Substrates

In addition to their harvested biomass being used as a feed additive, it is anticipated that cultures of tetrasporophytes can also be used in the preparation of seeded substrates when it is desired to establish offshore farms for the cultivation of a species of Asparagopsis. The terms “culture” and “cultivate” are used to distinguish between the onshore culture of tetrasporophytes and the offshore cultivation of gametophytes.

Cultures of tetrasporophytes of Asparagopsis armata maintained at 22 to 24° C. with a 12-hour alternating light-dark schedule with green light are induced to sporulate by reducing the temperature of the cultures to 12 to 14° C. and modifying the alternating light-dark schedule to 6 hours green light and 18 hours dark. The tetrasporophytes are induced to produce haploid tetraspores that readily adhere to a suitable substrate. For the purposes of establishing crops of haploid gametophytes for harvesting suitable substrates include biodegradable substrates such as cotton in the form of felted or woven material. Prior to transfer to the site of the offshore farm, the biodegradable substrate seeded with tetraspores is incubated until the early stages of haploid gametophyte development are observed. Once gametophyte development is established the seeded substrate is transferred to the site.

Although the invention has been described with reference to embodiments or examples it should be appreciated that variations and modifications may be made to these embodiments or examples without departing from the scope of the invention. Where known equivalents exist to specific elements, features or integers, such equivalents are incorporated as if specifically referred to in this specification. Variations and modifications to the embodiments or examples that include elements, features or integers disclosed in and selected from the referenced publications are within the scope of the invention unless specifically disclaimed. For example, it is anticipated that similar methods to those described here may be applied to the culture, harvesting and processing of biomass of Asparagopsis taxiformis. The advantages provided by the invention and discussed in the description may be provided in the alternative or in combination in these different embodiments of the invention.

### INDUSTRIAL APPLICABILITY

An animal feed additives and methods for the culture and cultivation of species of Asparagopsis used their preparation are provided. The additives can be included in the feed of ruminant animals to reduce methanogenesis.

### INCORPORATION BY REFERENCE

For the purposes of 37 C.F.R. 1.57 of the United States Code of Federal Regulations the disclosures of the following publications (as more specifically identified under the heading “Referenced Publications”) are incorporated by reference: Goldman and Mata (2021), Guillard and Ryther (1962), Harvey (1849) and Machado et al (2015).

### REFERENCED PUBLICATIONS

Anon (1994) Accuracy (trueness and precision) of measurement methods and results—Part 2: Basic method for the determination of repeatability and reproducibility of a standard measurement method ISO 5725-2:1994.
De Nys et al (2020) Novel composition International application no. PCT/AU2019/051335 [publ. no. WO 2020/113279 A1].
Dubois et al (2013) Effect of tropical algae as additives on rumen in vitro gas production and fermentation characteristics American Journal of Plant Sciences, 4, 34-43.
Goldman and Mata (2021) Bioreactor and method for culturing seaweed international application no. PCT/US2021/013744 [publ. no. WO 2021/150450 A1]
Guillard and Ryther (1962) Studies of marine planktonic diatoms. I. Cyclotella Nana Hustedt and Detonula Confervacea (Cleve) Gran. Canadian Journal of Microbiology, 8, 229.
Harvey (1849) Some account of the marine botany of the colony of Western Australia The Transactions of the Royal Irish Academy, 22, 525-566.
Lanigan (1972) Metabolism of pyrrolizidine alkaloids in the ovine rumen. IV. * Effects of chloral hydrate and halogenated methanes on rumen methanogenesis and alkaloid metabolism in fistulated sheep Aust. J. agric. Res., 23, 1085-91.
Lognone et al (2005) Method for the on-land production of red algae from the Bonnemaisoniaceae family International application no. PCT/FR2004/001825 [Publ. no. WO 2005/015983 A1].
Machado et al (2014) Effects of marine and freshwater macroalgae on in vitro total gas and methane production PLOS ONE 9(1): e85289.
Machado et al (2015) Method for reducing total gas production and/or methane production in a ruminant animal International application no. PCT/AU2015/000030 [WO 2015/109362 A2].
Paul et al (2006) Chemical defence against bacterial in the red alga Asparagopsis armata: linking structure with function Marine Ecology Progress Series 306, 87-101.
Sawant (2019) Novel crop fortification, nutrition and crop protection composition international application no. PCT/IB2018/055225 [publ. no. WO 2019/016661].
Tomkins et al (2009) A bromochloromethane formulation reduces enteric methanogenesis in cattle fed grain-based diets Animal Production Science, 49, 1053-1058.
Tomkins et al (2018) Growth performance improvements in pasture and feedlot systems international application no. PCT/AU2016/050689 [publ. no. WO 2018/018062 A1].

1. Harvested biomass of a species of Asparagopsis where the bromoform content of the biomass is greater than 7 mg/g wet weight.

2. The harvested biomass of claim 1 where the bromoform content of the biomass is greater than 14 mg/g wet weight.

3. The harvested biomass of claim 2 where the bromoform content of the biomass is greater than 21 mg/g wet weight.

4. The harvested biomass of claim 3 where the species of Asparagopsis is Asparagopsis armata.

5. The harvested biomass of claim 4 where the biomass consists essentially of tetrasporophytes.

6. An animal feed additive comprising the harvested biomass of claim 5.

7. A method of preparing an animal feed additive comprising harvesting biomass of a species of Asparagopsis and subjecting the harvested biomass to a physical stress that is sufficient to produce at least a two-fold increase in the bromoform content of the harvested biomass.

8. The method of claim 7 where the subjecting the biomass to a physical stress is sufficient to produce a change in colour of the biomass from red to purple.

9. The method of claim 8 where the physical stress comprises compression of the biomass, exposure of the biomass to sunlight, or partial desiccation of the biomass, or a combination of two or more thereof.

10. The method of claim 9 comprising:
(1) Incubating a circulating volume of seawater containing propagules of the species of Asparagopsis under green light to provide a culture;
(2) harvesting biomass from the culture;
(3) subjecting the biomass to at least partial desiccation so as to cause the biomass to develop a purple colour; and then
(4) freezing the biomass.

11. The method of claim 10 where the propagules of the species of Asparagopsis are tetrasporophytes.

12. The method of claim 11 where the species is Asparagopsis armata.
